(19) United States
(12) Patent Application Publication
(10) Pub. No.: US 2006/0168671 A1
(43) Pub. Date: Jul. 27, 2006

(54) Injection of caprine sperm factor (cSF), phospholipase C zeta (PLCzeta) and adenophostin A as alternative methods of activation during nuclear transfer in the caprine species

(76) Inventors: William G. Gavin, Dudley, MA (US); Teru Jellerette, Amherst, MA (US)

Correspondence Address: GTC BIOTHERAPEUTICS, INC., 175 CROSSING BOULEVARD, SUITE 410, FRAMINGHAM, MA, 01702, US

(21) Appl. No.: 11/040,271

(22) Filed: Jan. 21, 2005

(51) Int. Cl. A01K 67/027

(52) U.S. Cl. 800/14; 800/21

(57) ABSTRACT

It has been found that caprine sperm factor, alone or in combination with adenophostin A, produces the Ca²⁺ oscillations characteristic of egg activation seen with fertilization. The introduction of caprine sperm factor and adenophostin A to eggs to produce reconstructed embryos has been optimized.

### FIELD OF THE INVENTION

The present invention relates to improved methods for activation of reconstructed embryos for use in nuclear transfer procedures in non-human mammals. More specifically, the current invention provides a method to improve the activation of reconstructed embryos in nuclear transfer procedures in goats through the use of Caprine Sperm Factor, and/or Phospoholipase Cζ (PLCζ) and/or Adenophostin.

### BACKGROUND OF THE INVENTION

The present invention relates generally to the field of somatic cell nuclear transfer (SCNT) and to the creation of desirable transgenic animals. More particularly, it concerns methods for generating somatic cell-derived cell lines, transforming these cell lines, and using these transformed cells and cell lines to generate transgenic non-human mammalian animal species.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, are time-consuming, costly and unreliable. Moreover, these processes are completely incapable of allowing a specific animal line from producing gene products, such as desirable protein therapeutics that are otherwise entirely absent from the genetic complement of the species in question (i.e., spider silk proteins in bovine milk).

The development of technology capable of generating transgenic animals provides a means for exceptional precision in the production of animals that are engineered to carry specific traits or are designed to express certain proteins or other molecular compounds. That is, transgenic animals are animals that carry a gene that has been deliberately introduced into somatic and/or germline cells at an early stage of development. As the animals develop and grow the protein product or specific developmental change engineered into the animal becomes apparent.

At present the techniques available for the generation of transgenic domestic animals are inefficient and time-consuming typically producing a very low percentage of viable embryos. During the development of a transgene, DNA sequences are typically inserted at random, which can cause a variety of problems. The first of these problems is insertional inactivation, which is inactivation of an essential gene due to disruption of the coding or regulatory sequences by the incoming DNA. Another problem is that the transgene may either be not incorporated at all, or incorporated but not expressed. A further problem is the possibility of inaccurate regulation due to positional effects. This refers to the variability in the level of gene expression and the accuracy of gene regulation between different founder animals produced with the same transgenic constructs. Thus, it is not uncommon to generate a large number of founder animals and often confirm that less than 5% express the transgene in a manner that warrants the maintenance of the transgenic line.

At fertilization the sperm induces an increase in intracellular calcium that is essential for early and late events of egg activation. These events include cortical granule exocytosis, resumption of meiosis and the extrusion of the second polar body, as well as DNA synthesis, and the first mitotic cleavage (Kline and Kline, 1992). This increase in Ca²⁺ is commonly referred to as intracellular calcium oscillations ([Ca²⁺]i). The oscillation pattern is characterized by an initial large rise followed by additional smaller rises that decline in amplitude and frequency as time progresses. These oscillations can last from several minutes to several hours depending on the species in which they are initiated (Miyazaki et al., 1986). Further research shows that modulation of the [Ca²⁺]i pattern not only alters preimplantation events but postembryonic development in mammals (Ozil, 2001). Somatic cell research has shown that [Ca²⁺]i oscillations are essential for signaling gene expression and that changes in frequency of the oscillation alters what genes are expressed (Dolmetsch, et al., 1998).

The mechanism by which the sperm initiates these [Ca²⁺]i oscillations is not yet fully understood. However, it is widely supported that the sperm acts by stimulating the phosphoinositide (PI) pathway, resulting in the production of the signaling molecule inositol 1,4,5-trisphosphate (IP3), the binding of it to its receptor (IP3R) and the consequential release of calcium from intracellular stores in the endoplasmic reticulum (ER). Studies have been performed to parthenogenetically activate eggs by inducing an increase in [Ca²⁺]i, however several of these methods fall short of mimicking the patterning of oscillations seen at fertilization, resulting in only a single [Ca²⁺]i rise. These methods also require toxic chemicals to be applied such as ethanol, or a combination of ionomycin and 6-DMAP (Cuthbertson, 1983; Shiina et al., 1993).

Studies involving the microinjection of cytosolic sperm extracts or sperm factor (SF) into metaphase two (MII) stage eggs have shown [Ca²⁺]i oscillations similar to those seen at fertilization (Jones, et al., 1998), as well as high rates of activation to the blastocyst stage (Fissore, et al., 1998) and the ability to produce live young (Sakurai, et al., 1999). Studies show SF derived from porcine species is able to activate mouse MII eggs as well as bovine eggs (Stice, et al., 1990; Wu, et al., 1998) indicating that the factor is not species specific. Sperm factor has been shown to initiate fertilization-like oscillations by stimulating the PI pathway (Jones, et al., 1998). Research in purifying “Sperm Factor” has resulted in the partial characterization of its properties. Through column and sequencing studies, it has been established that SF consists of one or more sperm proteins found in the perinuclear theca of the mature spermatozoon (Perry, et al., 1999), however, the “factor(s)” have yet to be elucidated. Currently, a sperm specific protein known as phospholipase Cζ (PLCζ) has been identified and characterized (Saunders, et al. 2002). This PLCζ is believed to be the cytosolic sperm factor which has previously been investigated. Additionally, PLCζ has also been shown to initiate [Ca²⁺]i oscillations similar to those induced at fertilization by sperm and support subsequent early development (Cox, et al. 2002).

Other molecules have been used to parthenogenetically activate MII stage eggs by stimulating [Ca²⁺]i oscillations. Adenophostin A, a non-degradable IP3 analog induces fertilization-like oscillations and high activation rates in MII mouse eggs (Jellerette et al., 2000). This molecule is processed from the fungus Penicillium brevicompactum and is not degradable by the IP3 enzymes. It is the most potent known agonist of the IP3R (Takahashi et al., 1994), with a 10-100 fold greater affinity to IP3R than IP3 itself (Takahashi et al., 1994). This agonist has been shown to bind the IP3R at the IP3 binding site (Takahashi et al., 1994), causing egg activation and [Ca²⁺]i release similar to that seen at fertilization (Jellerette et al., 2000).

Currently the method of choice for activation during nuclear transfer (NT) is electrical pulsing, which induces a single [Ca²⁺]i rise. Recently, reports of studies in the equine and bovine species have shown that injection of cytosolic sperm extracts or sperm factor (SF) as an alternative method, resulting in higher activation numbers (Hindrichs et. al., 2001) and the ability to produce live young (Nott et al., 2002).

Thus, although transgenic animals have been produced by various methods in several different species, methods to readily and reproducibly produce transgenic animals capable of expressing the desired protein in high quantity or demonstrating the genetic change caused by the insertion of the transgene(s) at reasonable costs are still lacking.

Accordingly, a need exists for improved methods of nuclear transfer in the caprine and other ungulates that will allow an increase in production efficiencies in the development of transgenic animals, particularly with regard to the activation of fused cells during the simultaneous fusion and activation of cell couplets in an effort to produce viable transgenic offspring more reliably and efficiently.

### SUMMARY OF THE INVENTION

Briefly stated, the current invention provides a method for cloning a non-human mammal through an improved nuclear transfer process comprising: obtaining desired differentiated mammalian cells to be used as a source of donor nuclei; obtaining at least one oocyte from a mammal of the same species as the cells which are the source of donor nuclei; enucleating the at least one oocyte; transferring the desired differentiated cell or cell nucleus into the enucleated oocyte; simultaneously fusing and activating the cell couplet to form a first transgenic embryo, this process being done with the aid of caprine sperm factor, and/or phopholipase Cζ (PLCζ) and/or adenophostin A. That is, according to a preferred embodiment of the current invention mammalian egg activation is achieved by administering caprine sperm factor, PLCζ, adenophostin A, or any combination to eggs either just before, during, or just after nuclear transfer occurs to these eggs. The administration of one or any combination of these agents causes activation of the eggs to occur and further development of the animal to take place.

In particular, the methods of this invention are directed to the activation of caprine eggs. This species presents unique problems and opportunities for egg activation as animals are produced with specialized capabilities of producing identified, useful products.

Moreover, the method of the current invention also provides for optimizing the generation of transgenic animals through the use of caprine oocytes, arrested at the Metaphase-II stage, that were enucleated and fused with donor somatic cells and simultaneously activated.

It is also important to point out that the present invention can also be used to increase the availability of CICM cells, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimericism can be used to avoid immunological rejection among animals of the same species as well as between species.

### BRIEF DESCRIPTION OF THE DRAWINGS

FIG. 1 Shows A Generalized Diagram of the Process of Creating Cloned Animals through Nuclear Transfer.

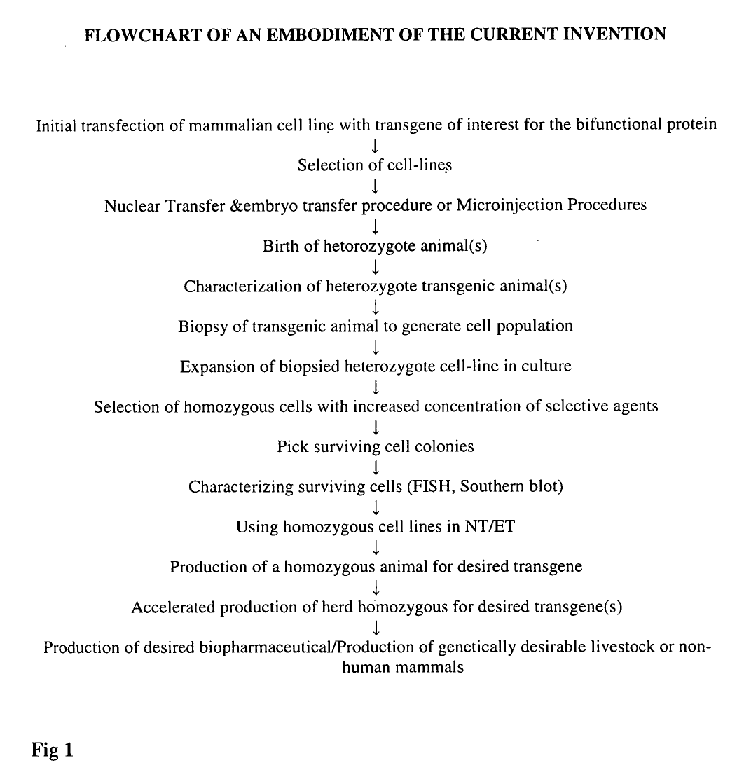

### DESCRIPTION OF THE PREFERRED EMBODIMENT

The following abbreviations have designated meanings in the specification:

Abbreviation Key:
Somatic Cell Nuclear Transfer (SCNT)
Cultured Inner Cell Mass Cells (CICM)
Nuclear Transfer (NT)
Synthetic Oviductal Fluid (SOF)
Fetal Bovine Serum (FBS)
Polymerase Chain Reaction (PCR)
Bovine Serum Albumin (BSA)
Phospholipase C zeta isoform (PLCζ)

Explanation of Terms:
Caprine—Of or relating to various species of goats.
Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.
Cell Couplet—An enucleated oocyte and a somatic or fetal karyoplast prior to fusion and/or activation.
Cytocholasin-B—A metabolic product of certain fungi that selectively and reversibly blocks cytokinesis while not effecting karyokinesis.
Cytoplast—The cytoplasmic substance of eukaryotic cells.
Karyoplast—A cell nucleus, obtained from the cell by enucleation, surrounded by a narrow rim of cytoplasm and a plasma membrane.
Somatic Cell—Any cell of the body of an organism except the germ cells.
Parthenogenic—The development of an embryo from an oocyte without the penetrance of sperm
Transgenic Organism—An organism into which genetic material from another organism has been experimentally transferred, so that the host acquires the genetic traits of the transferred genes in its chromosomal composition.

Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

The present invention relates to a system for an increasing the number of transgenic embryos developed for nuclear transfer procedures. The current invention provides an improved method for the creation of fused and activated embryos. This capability offers an improvement in the efficiency of the creation of activated and fused nuclear transfer-capable embryos for the production of live offspring in various mammalian non-human species including goats, pigs, rodents, primates, rabbits and cattle.

In addition, the present invention relates to cloning procedures in which cell nuclei derived from differentiated fetal or adult mammalian cells, which include non-serum starved differentiated fetal or adult caprine cells, are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce chimeric embryos, fetuses and/or offspring.

In mammals, while there are species differences, the initial signaling events and subsequent Ca⁺² oscillations induced by sperm at fertilization are the normal processes that result in oocyte activation and embryonic development (Fissore et al., 1992 and Alberio et al., 2001). Both chemical and electrical methods of Ca⁺² mobilization are currently utilized to activate couplets generated by somatic cell nuclear transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al., Biol. Reprod. 2001; Lai et al., 2001; Yong et al., 1998; and Kasinathan et al., Nature Biotech. 2001). However, there is quite a large degree of variability in the sequence, timing and methodology used for fusion and activation.

In order to mimic more closely the [Ca²⁺]i pattern induced by the sperm during fertilization, compositions are used comprising the more physiological agent caprine sperm factor (cSF). Compositions comprising cSF, PLCζ, adenophostin A are used, alone or in any combination, to provide an efficient method of activation. Caprine SF, PLCζ, adenophostin A, or any combination are introduced into NT caprine eggs prior to, simultaneously, or post reconstruction, providing sustained calcium release similar to that seen at fertilization. The compositions comprising cSF, PLCζ, adenophostin A, or any combination, are introduced into the reconstructed NT egg using injection or electrofusion. Due to the size of adenophostin A (660 kDa), experiments were performed comparing injection to electrofusion to incorporate the molecule into the reconstructed NT eggs. This will bypass the technically difficult microinjection technique, thereby removing a potentially invasive and harmful injection procedure.

PLCζ to be used in the method of the invention is in the RNA form and can be in the range of 0.1 mg/ml-10 mg/ml (concentration). According to the current invention, Adenophostin A to be used in the method of the invention can be in the range of 0.1 μM-100 μM. In a preferred embodiment when Adenophostin A is used in combination with cSF, the range of concentrations can again be 0.1 μM-100 μM.

“Isolated” as used herein means that the material is removed from its original environment (e.g., the environment in which it is naturally found). For example, a naturally-occurring protein present in a living animal, tissue, or cell is not isolated, but the same protein which is separated from some or all of the coexisting materials in the natural system, so that it is at least partially purified from other cell components, is isolated.

### Materials and Methods

SF Preparation

Sperm factor was prepared from caprine semen as previously described in Wu et al, 1998. Briefly, semen was washed twice with TL-Hepes medium, and pellet was resuspended in a solution containing 75 mM KCl, 20 mM Hepes, 1 mM ethylenediaminetetraacetic acid (EDTA), 10 mM glycerol phosphate, 1 mM dithiothreitol (DTT), 200 μM phenylmethanesulfonyl fluoride (PMSF), 10 μg/ml of pepstatin, and 10 μg/ml of leupeptin, pH 7.0. The resulting suspension was lysed by sonication for 30-35 min at 4° C. or by freezing and thawing for 5 min at −80° C. The lysate was then be centrifuged twice at 10 000×g, and the supernatants was collected and ultracentrifuged at 100 000×g for 1 hr at 4° C. The extracts will then be concentrated using ultrafiltration membranes (Centricon 30; Amicon, Beverly, Mass.). The crude sperm extracts was mixed with saturated ammonium sulfate to 50% saturation, then centrifuged at 10 000×g for 15 min at 4° C. The precipitates was collected and stored at −80° C. until use. Protein Concentration: Total protein concentration was 30 mg/ml. Source of adenophostin A (A.G. Scientific, San Diego, Calif.).

Egg Activation

Phase I:

Experiments in PHASE I will look at the quality of the cSF preparation as well as the ability of cSF and adenophostin A or a combination of both, to activate MII stage caprine oocytes. In vitro MII stage eggs was a) injected with 1, 2, or 5 mg/ml cSF, b) injected or c) electrofused with 20 μM adenophostin A (A.G. Scientific, San Diego, Calif.) or d) injected with a combination of the two agents. All eggs were monitored for activation and development to the blastocyst stage.

Phase I: Development (3 Replicates/Experiment)

| EXPERIMENT | PURPOSE | OOCYTE # |
|---|---|---|
| 1) SF concentration titration | | |
| 1.0 mg/ml | activation/dev | (3)20 |
| 2.0 mg/ml | activation/dev | (3)20 |
| 5.0 mg/ml | activation/dev | (3)20 |
| 2) Adenophostin A 20 μm | activation/dev | |
| injection | | (3)20 |
| fusion | | (3)20 |
| 3) Adenophostin | activation/dev | |
| injection with cSF | | (3)20 |
| fusion and injection of cSF | | (3)20 |
| Control: Iono/DMAP | oocyte quality (10/exp) | 210 |
| TOTAL | | 630 |

Experiments in PHASE I will look at the quality of the SF prep. as well as the ability of cSF and adenophostin A to activate MII stage caprine oocytes Development was assessed to the blastocyst stage, and compared to Iono/DMAP controls. The Iono/DMAP controls refer to the Applicants standard control activation protocol to assess oocyte quality. Pursuant to the current invention a combination of exposure to 5 μM ionomycin in TCM 199 w/10% FBS for 5 minutes and then cultured in TCM 199 w/10% FBS supplemented w/2 mM 6-dimethylaminopurine (DMAP) is used. All culturing was done in an incubator at 5% CO₂ in 100% humidity at 39 degrees Celsius.

Phase II:

Experiments in PHASE II. The ability of cSF and adenophostin A to activate NT eggs was investigated. Reconstructed NT eggs was injected with a) 5 mg/ml cSF and/or b) 20 μM adenophostin A at 30 min post fusion or 30 min post re-fusion c) 20 μM adenophostin A was added to the fusion buffer. The fusion buffer contains fusion buffer=0.3 M mannitol, 0.05 mM CaCl₂, 0.1 mM MgSO₄, 0.5 mM hepes and 0.3% BSA] for the first or second electropulsing.

Fusion and activation were performed at room temperature. Couplets were manually aligned equidistant between the electrodes of a 0.5 mm gap fusion chamber (Genetronics Biomedical, San Diego, Calif., USA) overlaid with fusion buffer (0.3 M mannitol, 0.05 mM CaCl₂, 0.1 mM MgSO₄, 0.5 mM hepes and 0.3% BSA). A single simultaneous fusion and activation electrical pulse of between 2.0 to 3.0 kV/cm for 20 μsec was applied to the couplets using a BTX ECM 2001 Electrocell Manipulator (Genetronics).] depending on the experiment performed. All eggs were monitored for activation and development. [Can you give criteria to define the success or failure of activation? . . . and for development?] Indication of activation and subsequent development in this part of the experiment is evaluated by cleavage in culture. Meaning, monitoring daily for development beyond the oocyte stage and onto a 2 cell, 4 cell, 8 cell, morula, blastocyst, etc. is the criteria.

Phase II: NT (3 Replicates/Experiment)

| EXPERIMENT | PURPOSE | OOCYTE # |
|---|---|---|
| 1) Simultaneous fusion and/or | activation of NT eggs | 3(30) |
| injection of adenophostin | | 3(30) |
| stimulation | | |
| 2) Injection of SF 30 min post | activation of NT eggs | 3(30) |
| fusion | | |
| 3) Injection of SF 30 min post | activation of NT eggs | 3(30) |
| re-fusion | | |
| Control: Iono/DMAP | oocyte quality (10/exp) | 120 |
| TOTAL | | 480 |

Phase III: PLCζ Injection.

Lastly, PLCζ will be assessed for its ability to support activation and development. This will be done through injection of PLCζ RNA into oocytes and monitoring of subsequent development in vitro as previously described.

Calcium Monitoring

Eggs activated with cSF or adenophostin A were monitored for [Ca²⁺]i oscillations. Monitoring of [Ca²⁺]i levels using Fura-2D-loaded eggs was carried out as previously described (Wu et al., 1998). For assessment of activation as well as for experiments looking at in vitro development, oocytes were observed under a phase contrast microscope at day two for cleavage to the two-cell stage as well as at day seven for blastocyst formation. In vivo eggs assessed as competent was transferred to recipient does and monitored for pregnancies.

Donor karyoplasts were obtained from a primary fetal somatic cell line derived from a 40-day transgenic female fetus produced by artificial insemination of a negative adult female with semen from a transgenic male. Through the methodology and system employed in the current invention transgenic animals, goats, were generated by somatic cell nuclear transfer and were shown to be capable of producing a target therapeutic protein in the milk of a cloned animal.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Oocyte Collection

Estrus synchronization and superovulation of donor does used as oocyte donors, and micro-manipulation was performed as described in Gavin W. G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Goats

The herds of pure- and mixed-breed scrapie-free Alpine, Saanen and Toggenburg dairy goats used for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses produced by artificially inseminating 2 non-transgenic female animals with fresh-collected semen from a transgenic male animal. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Sexing and Genotyping of Donor Cell Lines.

Genomic DNA was isolated from fetal tissue, and analyzed by polymerase chain reaction (PCR) for the presence of a target signal sequence, as well as, for sequences useful for sexing. The target transgenic sequence was detected by amplification of a 367-bp sequence. Sexing was performed using a zfX/zfY primer pair and Sac I restriction enzyme digest of the amplified fragments.

Preparation of Donor Cells for Embryo Reconstruction.

A transgenic female line (CFF6) was used for all nuclear transfer procedures. Fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 7 days. On the 7th day following the first addition of low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I.U. each/ml) 1 to 3 hours prior to fusion to the enucleated oocytes.

Cytoplast Preparation and Enucleation.

Oocytes with attached cumulus cells were discarded. Cumulus-free oocytes were divided into two groups: arrested Metaphase-II (one polar body) and Telophase-II protocols (no clearly visible polar body or presence of a partially extruding second polar body). The oocytes in the arrested Metaphase-II protocol were enucleated first. The oocytes allocated to the activated Telophase-II protocols were prepared by culturing for 2 to 4 hours in M199/10% FBS. After this period, all activated oocytes (presence of a partially extruded second polar body) were grouped as culture-induced, calcium-activated Telophase-II oocytes (Telophase-II-Ca) and enucleated. Oocytes that had not activated during the culture period were subsequently incubated 5 minutes in M199, 10% FBS containing 7% ethanol to induce activation and then cultured in M199 with 10% FBS for an additional 3 hours to reach Telophase-II (Telophase-II-EtOH protocol).

All oocytes were treated with cytochalasin-B (Sigma, 5 μg/ml in M199 with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. Telophase-II-Ca and Telophase-II-EtOH oocytes were enucleated by removing the first polar body and the surrounding cytoplasm (10 to 30% of cytoplasm) containing the partially extruding second polar body. After enucleation, all oocytes were immediately reconstructed.

Nuclear Transfer Embryo Culture and Transfer to Recipients.

All nuclear transfer embryos were co-cultured on monolayers of primary goat oviduct epithelial cells in 50 μl droplets of M199 with 10% FBS overlaid with mineral oil. Embryo cultures were maintained in a humidified 39° C. incubator with 5% CO₂ for 48 hours before transfer of the embryos to recipient does. Recipient embryo transfer was performed as previously described.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF₂α (Lutalyse, Upjohn). Parturition occurred within 24 hours after treatment. Kids were removed from the dam immediately after birth, and received heat-treated colostrum within 1 hour after delivery.

Genotyping of Cloned Animals.

Shortly after birth, blood samples and ear skin biopsies were obtained from the cloned female animals (e.g., goats) and the surrogate dams for genomic DNA isolation. Each sample was first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKem®, ME) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with α-³²P dCTP using the Prime-It® kit (Stratagene, La Jolla, Calif.). Hybridization was executed at 65° C. overnight. The blot was washed with 0.2×SSC, 0.1% SDS and exposed to X-OMAT™ AR film for 48 hours.

The present invention allows for increased efficiency of transgenic procedures by providing for an additional generation of activated and fused transgenic embryos. These embryos can be implanted in a surrogate animal or can be clonally propagated and stored or utilized. Also by combining nuclear transfer with the ability to modify and select for these cells in vitro, this procedure is more efficient than previous transgenic embryo techniques. According to the present invention, these transgenic cloned embryos can be used to produce CICM cell lines or other embryonic cell lines. Therefore, the present invention eliminates the need to derive and maintain in vitro an undifferentiated cell line that is conducive to genetic engineering techniques.

Thus, in one aspect, the present invention provides a method for cloning a mammal. In general, a mammal can be produced by a nuclear transfer process comprising the following steps:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo in the presence of caprine3 sperm factor, PLCζ, adenophostin A or any combination there of;
(vi) activating a cell-couplet that does not fuse to create a first transgenic embryo but that is activated after an initial electrical shock;
(vii) culturing said activated first and/or second transgenic embryo until greater than the 2-cell developmental stage; and
(viii) transferring said first and/or second transgenic embryo into a host mammal such that the embryo develops into a fetus.

Or,
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo in the presence of caprine sperm factor;
(vi) activating a cell-couplet that does not fuse to create a first transgenic embryo but that is activated after an initial electrical shock;
(vii) culturing said activated first and/or second transgenic embryo until greater than the 2-cell developmental stage; and
(viii) transferring said first and/or second transgenic embryo into a host mammal such that the embryo develops into a fetus.

Or,
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo in the presence of adenophostin A;
(vi) activating a cell-couplet that does not fuse to create a first transgenic embryo but that is activated after an initial electrical shock;
(vii) culturing said activated first and/or second transgenic embryo until greater than the 2-cell developmental stage; and
(viii) transferring said first and/or second transgenic embryo into a host mammal such that the embryo develops into a fetus.

Or,
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo in the presence of PLCζ;
(vi) activating a cell-couplet that does not fuse to create a first transgenic embryo but that is activated after an initial electrical shock;
(vii) culturing said activated first and/or second transgenic embryo until greater than the 2-cell developmental stage; and
(viii) transferring said first and/or second transgenic embryo into a host mammal such that the embryo develops into a fetus.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and offspring of those mammals. The present invention is preferably used for cloning caprines. The present invention further provides for the use of nuclear transfer fetuses and nuclear transfer and chimeric offspring in the area of cell, tissue and organ transplantation.

Also CICM cells derived from the methods described herein are advantageously used in the area of cell, tissue and organ transplantation, or in the production of fetuses or offspring, including transgenic fetuses or offspring. Differentiated mammalian cells are those cells, which are past the early embryonic stage. Differentiated cells may be derived from ectoderm, mesoderm or endoderm tissues or cell layers.

Mammalian cells, including human cells, may be obtained by well-known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures. Again the present invention is novel because differentiated cell types are used. The present invention is advantageous because the cells can be easily propagated, genetically modified and selected in vitro.

Suitable mammalian sources for oocytes include goats, sheep, cows, pigs, rabbits, guinea pigs, mice, hamsters, rats, primates, etc. Preferably, the oocytes will be obtained from caprines and ungulates, and most preferably goats. Methods for isolation of oocytes are well known in the art. Essentially, this will comprise isolating oocytes from the ovaries or reproductive tract of a mammal, e.g., a goat. A readily available source of goat oocytes is from hormonal induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields).

The stage of maturation of the oocyte at enucleation and nuclear transfer has been reported to be significant to the success of nuclear transfer methods. (First and Prather 1991). In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially goats, the oocyte activation period generally occurs at the time of sperm contact and penetrance into the oocyte plasma membrane.

After a fixed time maturation period, which ranges from about 10 to 40 hours, and preferably about 16-18 hours, the oocytes will be enucleated. Prior to enucleation the oocytes will preferably be removed and placed in EMCARE media containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in EMCARE media, preferably containing 7.5 micrograms per milliliter cytochalasin B, for immediate enucleation, or may be placed in a suitable medium, for example an embryo culture medium such as CR1aa, plus 10% FBS, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in EMCARE or SOF, and then viewing the oocytes under ultraviolet irradiation for less than 10 seconds. The oocytes that have been successfully enucleated can then be placed in a suitable culture medium.

In the present invention, the recipient oocytes will preferably be enucleated at a time ranging from about 10 hours to about 40 hours after the initiation of in vitro or in vivo maturation, more preferably from about 16 hours to about 24 hours after initiation of in vitro or in vivo maturation, and most preferably about 16-18 hours after initiation of in vitro or in vivo maturation.

Also, in some cases (e.g. with small donor nuclei) it may be preferable to inject the nucleus directly into the oocyte rather than using electroporation fusion. Such techniques are disclosed in Collas and Barnes, MOL. REPROD. DEV., 38:264-267 (1994), incorporated by reference in its entirety herein.

The activated embryo may be activated by known methods. Such methods include, e.g., culturing the activated embryo at sub-physiological temperature, in essence by applying a cold, or actually cool temperature shock to the activated embryo. This may be most conveniently done by culturing the activated embryo at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may best be effected by simultaneously, although protocols for sequential activation do exist. In terms of activation the following cellular events occur:
(i) increasing levels of divalent cations in the oocyte, and
(ii) reducing phosphorylation of cellular proteins in the oocyte.

Accordingly, it is to be understood that the embodiments of the invention herein providing for an increased availability of activated and fused “reconstructed embryos” are merely illustrative of the application of the principles of the invention. It will be evident from the foregoing description that changes in the form, methods of use, and applications of the elements of the disclosed method for the improved use of reconstructed embryos for SCNT are novel and may be modified and/or resorted to without departing from the spirit of the invention, or the scope of the appended claims.

### LITERATURE CITED AND INCORPORATED BY REFERENCE

1. Alberio R, et al., Mammalian Oocyte Activation: Lessons from the Sperm and Implications for Nuclear Transfer, INT J DEV BIOL 2001; 45: 797-809.
2. Alberio R, et al., Remodeling of Donor Nuclei, DNA Synthesis, and Ploidy of Bovine Cumulus Cell Nuclear Transfer Embryos: Effect of Activation Protocol, MOL REPROD DEV 2001; 59: 371-379.
3. Baguisi A, et al., Production of Goats by Somatic Cell Nuclear Transfer, NAT BIOTECH 1999; 17: 456-461.
4. Bondioli K, et al., Cloned Pigs from Cultured Skin Fibroblasts Derived from A H-Transferase Transgenic Boar, MOL REPROD DEV 2001; 60: 189-195.
5. Bondioli K R, et al., Production of Identical Bovine Offspring by Nuclear Transfer, THERIOGENOLOGY 1990; 33: 165-174.
6. Campbell, K H S, et al., Sheep Cloned by Nuclear Transfer From a Cultured Cell Line, NATURE 1996; 380: 64-66.
7. Cibelli J B, et al., Cloned Transgenic Calves Produced From Nonquiescent Fetal Fibroblasts. SCIENCE 1998; 280: 1256-1258.
8. Collas P, and Barnes F L., Nuclear Transplantation by Microinjection of Inner Cell Mass and Granulosa Cell Nuclei, MOL REPROD DEV. 1994 July; 38(3):264-7.
9. Cox L J, et al., Sperm phospholipase Cζ from humans and cytomologous monkeys triggers Ca 2+ oscillations, activation and development of mouse oocytes, REPROD 2002; 124: 611-623.
10. Cuthbertson, K. S. R. (1983). Parthenogenetic Activation of Mouse Oocytes In Vitro With Ethanol and Benzyl Alcohol, J. EXP. ZOOL. 226, 311-314.
11. Dolmetsch, R E., et al., Calcium Oscillations Increase The Efficiency And Specificity Of Gene Expression. NATURE 1998; 392, 933-936.
12. First N L, and Prather R S, Genomic Potential in Mammals, DIFFERENTIATION 1991 September; 48(1):1-8.
13. Fissore, R A., et al., (1998). Activation Of Development In Mammals: Is There A Role For Sperm Cytosolic Factor, THERIOGENOLOGY 49, 43-52.
14. Fissore R A, et al., Patterns of Intracellular Ca²⁺ Concentrations in Fertilized Bovine Eggs, BIOL REPROD 1992; 47: 960-969.
15. Gavin, W. G., Gene Transfer Into Goat Embryos, TRANSGENIC ANIMALS—GENERATION AND USE, L. M. Houdebine ed., (Harwood Academic Publishers Gmbh., 1996).
16. Groupen C G, et al., Activation of In Vivo and In Vitro Derived Porcine Oocytes by Using Multiple Electrical Pulses, REPROD FERT DEV 1999; 11: 457-462.
17. Jellerette, T., et al., (2000). Down-Regulation Of The Inositol 1,4,5-Trisphosphate Receptor In Mouse Eggs Following Fertilization Or Parthenogenetic Activation. DEV. BIOL. 223, 238-250.
18. Jones, K. T., et al., (1998). A Mammalian Sperm Cytosolic Phospholipase C Activity Generates Inositol Trisphosphate And Causes Ca²⁺ Release In Sea Urchin Egg Homogenates. FEBS LETT. 437, 297-300.
19. Kasinathan P, et al., Effect of Fibroblast Donor Cell Age and Cell Cycle on Development of Bovine Nuclear Transfer Embryos In Vitro, BIOL REPROD 2001; 64(5): 1487-1493.
20. Kasinathan P, et al., Production of Calves from G1 Fibroblasts, NATURE BIOTECH 2001; 19: 1176-1178.
21. Kato Y. et al., Cloning of Calves from Various Somatic Cell Types of Male and Female Adult, Newborn and Fetal Cows, J REPROD FERT 2000; 120: 231-237.
22. Kline, D., and Kline, J. T. (1992). Repetitive Calcium Transients And The Role Of Calcium In Exocytosis And Cell Cycle Activation In The Mouse Egg. DEV. BIOL. 149, 80-89.
23. Knott, J., et al., (2002). Porcine Sperm Factor Supports Activation And Development Of Bovine Nuclear Transfer Embryos. BIOL. REPROD. 66, 1095-1103.
24. Koo D B, et al., In Vitro Development Of Reconstructed Porcine Oocytes After Somatic Cell Nuclear Transfer. BIOL REPROD 2000; 63: 986-992.
25. Lai, L, et al., Feasibility of Producing Porcine Nuclear Transfer Embryos by Using G2/M-Stage Fetal Fibroblasts as Donors, BIOL REPROD 2001; 65: 1558-1564.
26. Meng L, Ely J J, Stouffer R L And D P Wolf, Rhesus Monkeys Produced by Nuclear Transfer, BIOL REPROD 1997; 57: 454-459.
27. Miyazaki, S., et al., (1986). Temporal And Spatial Dynamics Of The Periodic Increase In Intracellular Free Calcium At Fertilization Of Golden Hamster Eggs. J. CELL BIOL. 106, 345-353.
28. Ozil, J P., et al., (2001) Activation Of Rabbit Oocytes: The Impact Of The Ca²⁺ Signal Regime On Development. DEVELOPMENT 2001; 128:917-928.
29. Park K W, et al., Developmental Potential of Porcine Nuclear Transfer Embryos Derived from Transgenic Fibroblasts Infected with the Gene for the Green Fluorescent Protein: Comparison of Different Fusion/Activation Conditions, BIOL REPROD 2001; 65: 1681-1685.
30. Perry, A C F, et al., (1999). A Novel Trans-Complementation Assay Suggests Full Mammalian Oocyte Activation Is Coordinately Initiated By Multiple, Submembrane Sperm Components. BIOL. REPROD. 60, 747-755.
31. Polejaeva I A, et al., Cloned Pigs Produced by Nuclear Transfer from Adult Somatic Cells, NATURE 2000: 407: 505-509.
32. Ponimaskin E, et al., Sendai Virosomes Revisited: Reconstitution with Exogenous Lipids Leads to Potent Vehicles for Gene Transfer, VIROLOGY, 2000 Apr. 10; 269(2):391-403.
33. Sakurai, A., et al., Fertilization, Embryonic Development, And Offspring From Mouse Eggs Injected With Round Spermatids Combined With Calcium Oscillation-Inducing Sperm Factor. MOL. HUM. REPROD. 5, 132-138.
34. Saunders C M, et al., PLCζ: a sperm-specific trigger of Ca2+ oscillations in eggs and embryo development, DEVELOPMENT 2002; 129: 3533-3544.
35. Shiina, Y., et al., (1993). Role Of Extracellular Ca²⁺ In The Intracellular Ca²⁺ Changes In Fertilized And Activated Mouse Oocytes. J. REPROD. FERT. 97, 143-15.
36. Stice S L, et al., Pluripotent Bovine Embryonic Cell Lines Direct Embryonic Development Following Nuclear Transfer, BIOL REPROD. 1996 January; 54(1):100-10.
37. Stice S L and J M Robl, Nuclear Reprogramming in Nuclear Transplant Rabbit Embryo, BIOL REPROD 1992; 47: 636-643.
38. Stice, S L., and Robl, J M. (1990). Activation Of Mammalian Oocytes By A Factor Obtained From Rabbit Sperm. MOL. REPROD. DEV. 25, 272-280.
39. Takahashi, S., et al., (1994). Adenophostin A and B: Potent Agonists of Inositol-1,4,5-Trisphosphate Receptors Produced by Penicillin Bevicompactum. Structure Elucidation. J. ANTIBIOT. 47, 95-100.
40. Takahashi, M., et al., (1994). Adenophostins, Newly Discovered Metabolites of Penicillin Bevicompactum, Act As Potent Agonists Of The Inositol 1,4,5-Trisphosphate Receptor. J. BIOL. CHEM. 269, 369-372.
41. Wells D N, Misica P M And H R Tervit, Production of Cloned Calves Following Nuclear Transfer with Cultured Adult Mural Granulosa Cells, Biol Reprod 1999; 60: 996-1005.
42. Willadsen S M, Nuclear Transplantation in Sheep Embryos, NATURE 1986; 320: 63-65.
43. Wilmut I, et al., Viable Offspring Derived From Fetal and Adult Mammalian Cells. NATURE 1997; 385: 810-813.
44. Wu, H., et al., (1998). Injection Of Porcine Sperm Factor Induces Activation Of Mouse Eggs. Mol. REPROD. DEV. 49, 37-47.
45. Yang X, S Jiang, A Kovacs and R H Foote, Nuclear Totipotency of Cultured Rabbit Morulae to Support Full-Term Development Following Nuclear Transfer, BIOL REPROD 1992; 47: 636-643.
46. Yong Z And L Yuqiang, Nuclear-Cytoplasmic Interaction and Development of Goat Embryos Reconstructed by Nuclear Transplantation: Production of Goats by Serially Cloning Embryos, BIOL REPROD 1998; 58: 266-269.
47. Zou X, et al., Production of Cloned Goats from Enucleated Oocytes Injected with Cumulus Cell Nuclei or Fused with Cumulus Cells, CLONING 2001; 3 (1): 31-37.

What is claimed is:

1. A method for inducing caprine egg activation, said method comprising introducing a composition comprising isolated caprine sperm factor into one or more reconstructed caprine eggs.

2. The method of claim 1, wherein said donor cell or donor cell nucleus is from an ungulate selected from the group consisting of bovine, ovine, porcine, equine, caprine and buffalo.

3. The resultant offspring of the method of claim 1.

4. A method for inducing caprine egg activation, said method comprising introducing a composition comprising adenophostin A into one or more reconstructed caprine eggs.

5. A method for inducing caprine egg activation, said method comprising introducing a composition comprising isolated caprine sperm factor and adenophostin A into one or more reconstructed caprine eggs.

6. A method for inducing caprine egg activation, said method comprising introducing, simultaneously with nuclear transfer, a composition comprising isolated caprine sperm factor into one or more enucleated caprine eggs.

7. A method for inducing caprine egg activation, said method comprising introducing, simultaneously with nuclear transfer, a composition comprising adenophostin A into one or more enucleated caprine eggs.

8. A method for inducing caprine egg activation, said method comprising introducing, simultaneously with nuclear transfer, a composition comprising isolated caprine sperm factor and adenophostin A into one or more enucleated caprine eggs.

9. A method for inducing caprine egg activation, said method comprising introducing, preceding nuclear transfer, a composition comprising isolated caprine sperm factor into one or more enucleated caprine eggs.

10. A method for inducing caprine egg activation, said method comprising introducing, preceding nuclear transfer, a composition comprising adenophostin A into one or more enucleated caprine eggs.

11. The method of claim 10, wherein said donor cell or donor cell nucleus is from an ungulate selected from the group consisting of bovine, ovine, porcine, equine, caprine and buffalo.

12. A method for inducing caprine egg activation, said method comprising introducing, preceding nuclear transfer, a composition comprising isolated caprine sperm factor and adenophostin A into one or more enucleated caprine eggs.

13. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of caprine sperm factor;
(vi) culturing said activated transgenic embryo until greater than the 2-cell developmental stage; and
(vii) transferring said transgenic embryo into a host mammal such that the embryo develops into a fetus.

14. The method of claim 13, wherein said donor differentiated mammalian cell to be used as a source of donor nuclei or donor cell nucleus is from mesoderm.

15. The method of claim 13, wherein said donor differentiated mammalian cell to be used as a source of donor nuclei or donor cell nucleus is from endoderm.

16. The method of claim 13, wherein said donor differentiated mammalian cell to be used as a source of donor nuclei or donor cell nucleus is from ectoderm.

17. The method of claim 13, wherein said donor differentiated mammalian cell to be used as a source of donor nuclei or donor cell nucleus is from fetal somatic tissue.

18. The method of claim 13, wherein said donor differentiated mammalian cell to be used as a source of donor nuclei or donor cell nucleus is from fetal somatic cells.

19. The method of claim 13, wherein said donor differentiated mammalian cell to be used as a source of donor nuclei or donor cell nucleus is from a fibroblast.

20. The method of claim 13, wherein said donor differentiated mammalian cell to be used as a source of donor nuclei or donor cell nucleus is from an ungulate.

21. The method of claim 13, wherein said donor differentiated mammalian cell to be used as a source of donor nuclei or donor cell nucleus is from an adult non-human mammalian somatic cell.

22. The method of claim 13, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

23. The method of claim 13, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

24. The method of claim 13, wherein said at least one oocyte is matured in vivo prior to enucleation.

25. The method of claim 13, wherein said at least one oocyte is matured in vitro prior to enucleation.

26. The method of claim 13, wherein said non-human mammal is a rodent.

27. The method of claim 13, wherein said donor differentiated mammalian cell to be used as a source of donor nuclei or donor cell nucleus is a non-quiescent somatic cell or a nucleus isolated from said non-quiescent somatic cell.

28. The method of claim 13, wherein the fetus develops into an offspring.

29. The method of claim 13, wherein said at least one oocyte is enucleated about 10 to 60 hours after initiation of in vitro maturation.

30. The method of claim 13, wherein a desired gene is inserted, removed or modified in said differentiated mammalian cell or cell nucleus prior to insertion of said differentiated mammalian cell or cell nucleus into said enucleated oocyte.

31. The resultant offspring of the method of claim 30.

32. The method of claims 13, wherein cytocholasin-B is used in the cloning protocol.

33. The method of claim 13, wherein cytocholasin-B is not used in the cloning protocol.

34. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of Adenophostin A;
(vi) culturing said activated transgenic embryo until greater than the 2-cell developmental stage; and
(vii) transferring said transgenic embryo into a host mammal such that the embryo develops into a fetus.

35. The method of claim 34, wherein said donor differentiated mammalian cell to be used as a source of donor nuclei or donor cell nucleus is from mesoderm.

36. The method of claim 34, wherein said donor differentiated mammalian cell to be used as a source of donor nuclei or donor cell nucleus is from endoderm.

37. The method of claim 34, wherein said donor differentiated mammalian cell to be used as a source of donor nuclei or donor cell nucleus is from ectoderm.

38. The method of claim 34, wherein said donor differentiated mammalian cell to be used as a source of donor nuclei or donor cell nucleus is from fetal somatic tissue.

39. The method of claim 34, wherein said donor differentiated mammalian cell to be used as a source of donor nuclei or donor cell nucleus is from fetal somatic cells.

40. The method of claim 34, wherein said donor differentiated mammalian cell to be used as a source of donor nuclei or donor cell nucleus is from a fibroblast.

41. The method of claim 34, wherein said donor differentiated mammalian cell to be used as a source of donor nuclei or donor cell nucleus is from an ungulate.

42. The method of claim 34, wherein said donor differentiated mammalian cell to be used as a source of donor nuclei or donor cell nucleus is from an adult non-human mammalian somatic cell.

43. The method of claim 34, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

44. The method of claim 34, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

45. The method of claim 34, wherein said at least one oocyte is matured in vivo prior to enucleation.

46. The method of claim 34, wherein said at least one oocyte is matured in vitro prior to enucleation.

47. The method of claim 34, wherein said donor differentiated mammalian cell to be used as a source of donor nuclei or donor cell nucleus is a non-quiescent somatic cell or a nucleus isolated from said non-quiescent somatic cell.

48. The method of claim 34, wherein said non-human mammal is a rodent.

49. The method of claim 34, wherein the fetus develops into an offspring.

50. The method of claim 34, wherein a desired gene is inserted, removed or modified in said differentiated mammalian cell or cell nucleus prior to insertion of said differentiated mammalian cell or cell nucleus into said enucleated oocyte.

51. The method of claim 34, wherein said at least one oocyte is enucleated about 10 to 60 hours after initiation of in vitro maturation.

52. The resultant offspring of the method of claim 51.

53. The method of claim 34, wherein cytocholasin-B is not used in the cloning protocol.

54. The method of claims 34, wherein cytocholasin-B is used in the cloning protocol.

55. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of Adenophostin A and caprine sperm factor;
(vi) culturing said activated transgenic embryo until greater than the 2-cell developmental stage; and
(vii) transferring said transgenic embryo into a host mammal such that the embryo develops into a fetus.

56. The method of claim 55, wherein said donor differentiated mammalian cell to be used as a source of donor nuclei or donor cell nucleus is from mesoderm.

57. The method of claim 55, wherein said donor differentiated mammalian cell to be used as a source of donor nuclei or donor cell nucleus is from endoderm.

58. The method of claim 55, wherein said donor differentiated mammalian cell to be used as a source of donor nuclei or donor cell nucleus is from ectoderm.

59. The method of claim 55, wherein said donor differentiated mammalian cell to be used as a source of donor nuclei or donor cell nucleus is from fetal somatic tissue.

60. The method of claim 55, wherein said donor differentiated mammalian cell to be used as a source of donor nuclei or donor cell nucleus is from fetal somatic cells.

61. The method of claim 55, wherein said donor differentiated mammalian cell to be used as a source of donor nuclei or donor cell nucleus is from a fibroblast.

62. The method of claim 55, wherein said donor differentiated mammalian cell to be used as a source of donor nuclei or donor cell nucleus is from an ungulate.

63. The method of claim 55, wherein said donor differentiated mammalian cell to be used as a source of donor nuclei or donor cell nucleus is from an adult non-human mammalian somatic cell.

64. The method of claim 55, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

65. The method of claim 55, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

66. The method of claim 55, wherein said at least one oocyte is matured in vivo prior to enucleation.

67. The method of claim 55, wherein said at least one oocyte is matured in vitro prior to enucleation.

68. The method of claim 55, wherein said non-human mammal is a rodent.

69. The method of claim 55, wherein said donor differentiated mammalian cell to be used as a source of donor nuclei or donor cell nucleus is a non-quiescent somatic cell or a nucleus isolated from said non-quiescent somatic cell.

70. The method of claim 55, wherein the fetus develops into an offspring.

71. The method of claim 55, wherein said at least one oocyte is enucleated about 10 to 60 hours after initiation of in vitro maturation.

72. The method of claim 55, wherein a desired gene is inserted, removed or modified in said differentiated mammalian cell or cell nucleus prior to insertion of said differentiated mammalian cell or cell nucleus into said enucleated oocyte.

73. The resultant offspring of the methods of claim 72.

74. The method of claim 55, wherein cytocholasin-B is used in the cloning protocol.

75. The method of claim 55, wherein cytocholasin-B is not used in the cloning protocol.

76. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of PLCζ;
(vi) culturing said activated transgenic embryo until greater than the 2-cell developmental stage; and
(vii) transferring said transgenic embryo into a host mammal such that the embryo develops into a fetus.

77. The method of claim 76, wherein said donor differentiated mammalian cell to be used as a source of donor nuclei or donor cell nucleus is from mesoderm.

78. The method of claim 76, wherein said donor differentiated mammalian cell to be used as a source of donor nuclei or donor cell nucleus is from endoderm.

79. The method of claim 76, wherein said donor differentiated mammalian cell to be used as a source of donor nuclei or donor cell nucleus is from ectoderm.

80. The method of claim 76, wherein said donor differentiated mammalian cell to be used as a source of donor nuclei or donor cell nucleus is from fetal somatic tissue.

81. The method of claim 76, wherein said donor differentiated mammalian cell to be used as a source of donor nuclei or donor cell nucleus is from fetal somatic cells.

82. The method of claim 76, wherein said donor differentiated mammalian cell to be used as a source of donor nuclei or donor cell nucleus is from a fibroblast.

83. The method of claim 76, wherein said donor differentiated mammalian cell to be used as a source of donor nuclei or donor cell nucleus is from an ungulate.

84. The method of claim 76, wherein said donor differentiated mammalian cell to be used as a source of donor nuclei or donor cell nucleus is from an adult non-human mammalian somatic cell.

85. The method of claim 76, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

86. The method of claim 76, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

87. The method of claim 76, wherein said at least one oocyte is matured in vivo prior to enucleation.

88. The method of claim 76, wherein said at least one oocyte is matured in vitro prior to enucleation.

89. The method of claim 76, wherein said non-human mammal is a rodent.

90. The method of claim 76, wherein said donor differentiated mammalian cell to be used as a source of donor nuclei or donor cell nucleus is a non-quiescent somatic cell or a nucleus isolated from said non-quiescent somatic cell.

91. The method of claim 76, wherein the fetus develops into an offspring.

92. The method of claim 76, wherein said at least one oocyte is enucleated about 10 to 60 hours after initiation of in vitro maturation.

93. The method of claim 76, wherein a desired gene is inserted, removed or modified in said differentiated mammalian cell or cell nucleus prior to insertion of said differentiated mammalian cell or cell nucleus into said enucleated oocyte.

94. The resultant offspring of the methods of claim 93.

95. The method of claim 76, wherein cytocholasin-B is used in the cloning protocol.

96. The method of claim 76, wherein cytocholasin-B is not used in the cloning protocol.

97. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of Adenophostin A and PLCζ;
(vi) culturing said activated transgenic embryo until greater than the 2-cell developmental stage; and
(vii) transferring said transgenic embryo into a host mammal such that the embryo develops into a fetus.

98. The method of claim 97, wherein said donor differentiated mammalian cell to be used as a source of donor nuclei or donor cell nucleus is from mesoderm.

99. The method of claim 97, wherein said donor differentiated mammalian cell to be used as a source of donor nuclei or donor cell nucleus is from endoderm.

100. The method of claim 97, wherein said donor differentiated mammalian cell to be used as a source of donor nuclei or donor cell nucleus is from ectoderm.

101. The method of claim 97, wherein said donor differentiated mammalian cell to be used as a source of donor nuclei or donor cell nucleus is from fetal somatic tissue.

102. The method of claim 97, wherein said donor differentiated mammalian cell to be used as a source of donor nuclei or donor cell nucleus is from fetal somatic cells.

103. The method of claim 97, wherein said donor differentiated mammalian cell to be used as a source of donor nuclei or donor cell nucleus is from a fibroblast.

104. The method of claim 97, wherein said donor differentiated mammalian cell to be used as a source of donor nuclei or donor cell nucleus is from an ungulate.

105. The method of claim 97, wherein said donor differentiated mammalian cell to be used as a source of donor nuclei or donor cell nucleus is from an adult non-human mammalian somatic cell.

106. The method of claim 97, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

107. The method of claim 97, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

108. The method of claim 97, wherein said at least one oocyte is matured in vivo prior to enucleation.

109. The method of claim 97, wherein said at least one oocyte is matured in vitro prior to enucleation.

110. The method of claim 97, wherein said non-human mammal is a rodent.

111. The method of claim 97, wherein said donor differentiated mammalian cell to be used as a source of donor nuclei or donor cell nucleus is a non-quiescent somatic cell or a nucleus isolated from said non-quiescent somatic cell.

112. The method of claim 97, wherein the fetus develops into an offspring.

113. The method of claim 97, wherein said at least one oocyte is enucleated about 10 to 60 hours after initiation of in vitro maturation.

114. The method of claim 97, wherein a desired gene is inserted, removed or modified in said differentiated mammalian cell or cell nucleus prior to insertion of said differentiated mammalian cell or cell nucleus into said enucleated oocyte.

115. The resultant offspring of the methods of claim 114.

116. The method of claim 97, wherein cytocholasin-B is used in the cloning protocol.

117. The method of claim 97, wherein cytocholasin-B is not used in the cloning protocol.

118. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo in the presence of PLCζ and caprine sperm factor;
(vi) culturing said activated transgenic embryo until greater than the 2-cell developmental stage; and
(vii) transferring said transgenic embryo into a host mammal such that the embryo develops into a fetus.

119. The method of claim 118, wherein said donor differentiated mammalian cell to be used as a source of donor nuclei or donor cell nucleus is from mesoderm.

120. The method of claim 118, wherein said donor differentiated mammalian cell to be used as a source of donor nuclei or donor cell nucleus is from endoderm.

121. The method of claim 118, wherein said donor differentiated mammalian cell to be used as a source of donor nuclei or donor cell nucleus is from ectoderm.

122. The method of claim 118, wherein said donor differentiated mammalian cell to be used as a source of donor nuclei or donor cell nucleus is from fetal somatic tissue.

123. The method of claim 118, wherein said donor differentiated mammalian cell to be used as a source of donor nuclei or donor cell nucleus is from fetal somatic cells.

124. The method of claim 118, wherein said donor differentiated mammalian cell to be used as a source of donor nuclei or donor cell nucleus is from a fibroblast.

125. The method of claim 118, wherein said donor differentiated mammalian cell to be used as a source of donor nuclei or donor cell nucleus is from an ungulate.

126. The method of claim 118, wherein said donor differentiated mammalian cell to be used as a source of donor nuclei or donor cell nucleus is from an adult non-human mammalian somatic cell.

127. The method of claim 118, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

128. The method of claim 118, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

129. The method of claim 118, wherein said at least one oocyte is matured in vivo prior to enucleation.

130. The method of claim 118, wherein said at least one oocyte is matured in vitro prior to enucleation.

131. The method of claim 118, wherein said non-human mammal is a rodent.

132. The method of claim 118, wherein said donor differentiated mammalian cell to be used as a source of donor nuclei or donor cell nucleus is a non-quiescent somatic cell or a nucleus isolated from said non-quiescent somatic cell.

133. The method of claim 118, wherein the fetus develops into an offspring.

134. The method of claim 118, wherein said at least one oocyte is enucleated about 10 to 60 hours after initiation of in vitro maturation.

135. The method of claim 118, wherein a desired gene is inserted, removed or modified in said differentiated mammalian cell or cell nucleus prior to insertion of said differentiated mammalian cell or cell nucleus into said enucleated oocyte.

136. The resultant offspring of the method of claim 135.

137. The method of claim 118, wherein cytocholasin-B is used in the cloning protocol.

138. The method of claim 118, wherein cytocholasin-B is not used in the cloning protocol.
